(19) United States
(12) Patent Application Publication
(10) Pub. No.: US 2022/0143131 A1
(43) Pub. Date: May 12, 2022

(54) Compositions and Methods for increasing Reproduction Performance in Non Human Mammals Using Recombinant Luteinizing Hormone

(71) Applicant: Ceva Sante Animale, Libourne (FR)

(72) Inventors: Alessio Valenza, Libourne (FR); Alexandre Souza, Libourne (FR); Mark Colgin, Libourne (FR)

(21) Appl. No.: 17/611,660

(22) PCT Filed: May 18, 2020
(86) PCT No.: PCT/EP2020/063799

§ 371 Date: Nov. 16, 2021

(30) Foreign Application Priority Data
May 16, 2019 (EP) 19305630.6

(51) Int. Cl. A61K 38/09; A61K 31/5575; A61K 31/565; A61P 15/08

(52) U.S. Cl. CPC A61K 38/09; A61P 15/08; A61K 31/565; A61K 31/5575

(57) ABSTRACT

The present invention relates to methods and compositions for increasing reproduction performance in non-human mammals using recombinant luteinizing hormone (rLH) in a low dose. The invention also relates to methods for increasing follicle growth rates at later stages of synchronization programs, improving ovulation results, corpus luteum (CL) development after ovulation, or pregnancies in non-human mammal using rLH in a low dose. The invention is preferably used in ungulates such as bovine, in association to synchronization programs for timed ovulation.

The present invention relates to methods and compositions for increasing reproduction performance in non-human mammals using recombinant luteinizing hormone (rLH). The invention also relates to methods for increasing follicle growth rates at later stages of synchronization programs, improving ovulation results, corpus luteum (CL) development after ovulation, or pregnancies in non-human mammal using recombinant luteinizing hormone (rLH) in a low dose. The invention is preferably used in ungulates such as bovine, in association to synchronization programs for timed ovulation.

### BACKGROUND OF THE INVENTION

Synchronization protocols involving a sequence of reproductive hormones that cause an ovulation at a predicted time have been developed in the last decades to facilitate cattle breeding. Nowadays the utilization of these synchronization programs, also called “timed-artificial insemination” (TAI), protocols are widespread and utilized in most commercial beef and dairy herds.

The ability to increase reproductive performance in non-human mammals such as cattle, horses or other ungulates can have a significant benefit to farm owners. This can be achieved through increasing fertility and/or fecundity in such non-human mammals. Currently, several methods or protocols are proposed to increase reproductive performance, based on the use of estradiol compounds and other hormones as GnRH and PgF_{2α}, to mimic natural or close to natural levels of hormones occurring at the target specie. These protocols generally comprise steps to synchronize ovulation in females, to facilitate stimulation, growth and ovulation of follicles in a synchronized fashion allowing the fixed-time artificial insemination, avoiding the necessity of estrus detection. These so called synchronization protocols are widely used in commercial dairy and beef herds worldwide.

Adoption of the TAI technology is mainly driven by inefficient estrus detection in both beef and dairy herds, although the underlying reason for poor rates of estrus detection differ a lot across production systems and animal category. For example, in beef herds, the main drawback to adequate estrus detection is the lactation-induced anestrus caused by inhibition of gonadotropin release due to calf suckling in the postpartum period; whereas, for high producing dairy cows the low estrus detection efficiency is mainly due to high rates of steroid hormone metabolism by the liver, which in turn cause a drastic reduction of reproductive hormones in blood stream limiting estrus activity in the modern dairy cow (Yavas, Y., Walton, J. S., 2000. Postpartum acyclicity in suckled beef cows: A review. Theriogenology 54, 25-55, 2000; Wiltbank, M. C., Gümen, A., Sartori, R., 2002. Physiological classification of anovulatory conditions in cattle. Theriogenology 57, 21-52).

Postpartum anestrus in beef cattle has been managed over the years through several strategies including calf-removal at strategic times in the postpartum period to induce cyclicity (Dunn Jr, R. T. et al., 1985. Effects of 72 hr calf removal and/or gonadotropin releasing hormone on luteinizing hormone release and ovarian activity in postpartum beef cows. Theriogenology 23, 767-776). Once calves are removed at least temporarily from their dams there is a sudden increase in LH pulses culminating with ovulation in a matter of days. Briefly, calf-removal will decrease the amount of endogenous opioid in the cow inducing greater GnRH and LH/FSH release. Once TAI became more common in beef herds, producers and technicians started to associate calf-removal towards the end of the TAI protocol to maintain greater ovarian follicle growth and maximize ovulation as well as conception results. Some of the concerns of the temporary calf-removal are related to the difficulty to manage cows and calves once they are separate—not all herds have facilities that allow this type of management and some cows may reject their calves once they are reunited—this is particularly a problem in primiparous cows. Furthermore, calf stress in the case of heavy rain or food restriction during the separation days may trigger diseases in calves such as diarrhea or respiratory problems. Ultimately there are a number of cow-calf welfare issues involved in the calf removal management that requires attention.

Because of the complications related to calf-removal, modern synchronization protocols in beef cattle and mixed breeds utilize equine gonadotropin (eCG) instead of applying the calf removal management towards the end of the progesterone-based protocol. eCG is a glycoprotein hormone that has a long half-life (3 to 4 days in the cow) with both FSH and LH-like activity. eCG is able to bind and activate gonadotropin receptors in the growing follicle in cattle to hasten and maintain its growth until timed ovulation occurs (Murphy, B., 2012. Equine chorionic gonadotropin: an enigmatic but essential tool. Anim Reprod 9, 223-230). Although results following the use of eCG associated with timed-AI protocols are significantly improved compared to no-treatment (Baruselli et al., 2004 The use of hormonal treatments to improve reproductive performance of anestrous beef cattle in tropical climates. Animal Reproduction Science 82-83, 479-486), eCG is currently extracted from pregnant mares and lately there has been significant criticism related to its production utilizing frequent blood sampling from pregnant mares. Altogether, the complexity linked to collection and extraction of eCG, its high relative costs, and the pressure from public opinion on animal welfare are making the use of eCG in TAI protocols prohibitive. That has already led animal health companies to remove eCG from their portfolio in some countries. Thus, alternatives to eCG are of high priority, in particular when it comes to synchronization programs for beef cattle or even dairy cattle kept in pasture systems.

Some of the possible alternatives to eCG would include products already in the market such as gonadotropin releasing hormone (GnRH) and human chorionic gonadotropin (hCG). GnRH is somewhat limiting because it triggers a quick FSH/LH surge, however its follicle stimulating effects would last no longer than 4 to 5 hours, which would require multiple treatments throughout 2 to 3 days making it impossible in practical terms for a cattle herd. hCG instead, has a long-relative half-life maintaining its biological effects in the cow for more than 30 h due to its highly glycosylated molecule (Stevenson, J., et al., 2007. Interventions After Artificial Insemination: Conception Rates, Pregnancy Survival, and Ovarian Responses to Gonadotropin-Releasing Hormone, Human Chorionic Gonadotropin, and Progesterone. Journal of dairy science 90, 331-340; Giordano, J. et al., 2013. Ovulatory follicle dysfunction in lactating dairy cows after treatment with Folltropin-V at the onset of luteolysis. Theriogenology 79, 1210-1217). Unlike eCG, that presents both FSH and LH activities, hCG does not have FSH-like activity and only has LH-like effects. Because of its singular activity, it is generally utilized to induce ovulation in cystic animals (De Rensis, F. et al., 2008. Reproductive performance of dairy cows with ovarian cysts after synchronizing ovulation using GnRH or hCG during the warm or cool period of the year. Theriogenology 69, 481-484), or during synchronization protocols to induce ovulation (Keskin, A. et al., 2010. Effect of hCG vs. GnRH at the beginning of the Ovsynch on first ovulation and conception rates in cyclic lactating dairy cows. Theriogenology 74, 602-607) and/or to create accessory corpora lutea (CL) (Nascimento et al., 2012, J. Dairy Sci. 96:2873-2882) after ovulation, instead of being used to maintain follicular growth.

In cattle, the dominant follicle acquires an abundance of LH receptors soon after the deviation phase in the follicular wave. It could imply that LH alone could activate its receptors and stimulate follicle growth. However, previous research (Prata, A. B. et al., 2018. Effect of different chorionic gonadotropins on final growth of the dominant follicle in Bos indicus cows. Theriogenology 111, 52-55) failed to demonstrate that hCG (with its LH-like activity) could be used as a replacement of eCG during synchronization protocols; Prata et al., 2018 reported from ˜12 to 44% of premature ovulations in a dose-dependent fashion when utilizing 200 to 300 IU given at multiple times at later stages of the synchronization protocol.

A recombinant luteinizing hormone was studied and described in the patent application WO2004/078061. It was demonstrated that rbLH (at the dose of 1 mg or 2 mg) could significantly improve ovulation rate in high level of progesterone compared to the current available GnRH. The higher ovulatory response of rbLH in comparison to GnRH is due to the affinity of the molecule to bind the LH receptors on the follicle and the longer biological activity than the natural LH (˜30 h).

The goal of the invention is to provide an alternative to eCG in synchronization protocols resulting in a satisfactory reproductive performance, including improvement of fecundity and/or pregnancy rate, by maintaining follicle growth. In that respect, the inventors have found that a low dose of recombinant luteinizing hormone (rLH) as a single treatment at later stages of the synchronization protocol and before insemination can fulfil that objective.

### SUMMARY OF THE INVENTION

The present invention relates to methods and compositions for increasing reproductive performance in a non-human mammal using a recombinant luteinizing hormone in a dose ranging from about 50 to 300 micrograms of per mammal. The present invention also provides methods and compositions for increasing follicle growth rates at later stages of synchronization programs, improving ovulation results, corpus luteum (CL) development after ovulation, or pregnancies in non-human mammal using a recombinant luteinizing hormone in a specific dose range. The present invention allows a satisfactory reproduction, particularly fertility and fecundity, in non-human mammals.

More particularly, the invention provides improved rLH-based compositions and treatment methods which provide improved reproductive performance in non-human mammals by minimizing the dose to be administered as well as number of interventions (single treatment), thereby maintaining follicle growth and possibly avoiding premature ovulation.

A particular object of the invention relates to a rLH or to a composition comprising rLH, for use for increasing reproductive performance in non-human mammals undergoing a timed-artificial insemination protocol and/or treatment, where the rLH is administered at a dose range comprised between about 50 and about 300 micrograms, preferably with a simultaneous administration of estradiol and/or PGF2a compound. In a more particular object of the invention, the rLH is administered at a dose range comprised between 50 and 150 micrograms, preferably with a simultaneous administration of estradiol and/or PGF2a compound. In an even more particular object of the invention, the rLH is administered at a dose of 50, 100 or 150 micrograms, preferably with a simultaneous administration of estradiol and/or PGF2a compound.

Another object of the invention resides in rLH (or a composition comprising rLH) for use to increase reproductive performance in one or several non-human mammals, wherein rLH is administered to each of said one or several non-human mammals, at a dose range comprised between about 50 and about 300 micrograms, preferably with a simultaneous administration of estradiol and/or PGF2a compound. A more specific object of the invention resides in rLH (or a composition comprising rLH) for use to increase reproductive performance in one or several non-human mammals, wherein rLH is administered to each of said one or several non-human mammals, at a dose range comprised between about 50 and about 150 micrograms, preferably with a simultaneous administration of estradiol and/or PGF2a compound.

Another object of the invention resides in a method for increasing reproductive performance in one or several non-human mammals, comprising administering to each of said one or several non-human mammals rLH at a dose range comprised between about 50 and about 300 micrograms, preferably with a simultaneous administration of estradiol and/or PGF2a compound. A more specific object of the invention resides in a method for increasing reproductive performance in one or several non-human mammals, comprising administering to each of said one or several non-human mammals rLH at a dose range comprised between about 50 and about 150 micrograms, preferably with a simultaneous administration of estradiol and/or PGF2a compound.

Another object of the invention is a method for increasing ovulation and/or fertility and/or fecondity rate in one or several non-human mammals, comprising administering to each of said one or several non-human mammals rLH at a dose range comprised between about 50 and about 300 micrograms, preferably with a simultaneous administration of estradiol and/or PGF2a compound. A more specific object of the invention is a method for increasing ovulation and/or fertility and/or fecundity rate in one or several non-human mammals, comprising administering to each of said one or several non-human mammals rLH at a dose range comprised between about 50 and about 150 micrograms (such as 100 micrograms), preferably with a simultaneous administration of estradiol and/or PGF2a compound.

Another object of the invention is an improved method for producing or recovering oocytes in one or several non-human mammals, comprising administering to each of said one or several non-human mammals rLH at a dose range comprised between about 50 and about 300 micrograms, preferably with a simultaneous administration of estradiol and/or PGF2a compound. A more specific object of the invention is a method for producing or recovering oocytes in one or several non-human mammals, comprising administering to each of said one or several non-human mammals rLH at a dose range comprised between about 50 and about 150 micrograms (such as 100 micrograms), preferably with a simultaneous administration of estradiol and/or PGF2a compound.

Another object of the invention is a method for increasing the size of corpora lutea (CL) in a non-human mammal, comprising administering to said non-human mammal rLH at a dose range comprised between about 50 and about 300 micrograms, preferably with a simultaneous administration of estradiol and/or PGF2a compound.

A more specific object of the invention is a method for increasing the size of corpora lutea (CL) in a non-human mammal, comprising administering to said non-human mammal rLH at a dose range comprised between about 50 and about 150 micrograms (such as 100 micrograms), preferably with a simultaneous administration of estradiol and/or PGF2a compound.

According to the invention, administration of rLH is preferably performed in one single administration. Ideally performed at the time of progesterone device removal, and in case of fixed-time artificial insemination, breeding needs to occur ideally from 36 to 40 h after hCG treatment that took place at device removal.

In a particular embodiment, the invention relates to a method comprising:

(a) providing a non-human mammal (e.g., an ungulate), or a group of non-human mammals (e.g., of ungulates), which has been treated for a timed-artificial insemination protocol and/or treatment; or treating a non-human mammal (e.g., an ungulate), or a group of non-human mammals (e.g. of ungulates), for a timed-artificial insemination protocol and/or treatment;

(b) administering to said non-human mammal or group of mammals rLH at a dose range comprised between about 50 and about 300 micrograms, preferably with a simultaneous administration of estradiol and/or PGF2a compound, preferably by a single injection, more preferably by intramuscular or sub-cutaneous injection; and optionally

(c) inseminating the non-human mammal or group of non-human mammals, preferably by artificial insemination, preferably near the time of ovulation and, more preferably, 1 to 4 days, even more preferably 36 to 48 hours after administration step (b).

In a more particular embodiment of the invention, the invention relates to a method comprising:

(a) providing a non-human mammal (e.g., an ungulate), or a group of non-human mammals (e.g., of ungulates), which has been treated for a timed-artificial insemination protocol and/or treatment; or treating a non-human mammal (e.g., an ungulate), or a group of non-human mammals (e.g. of ungulates), for a timed-artificial insemination protocol and/or treatment;

b) administering to said non-human mammal or group of mammals rLH at a dose range comprised between about 50 and about 150 micrograms, preferably with a simultaneous administration of estradiol and/or PGF2a compound, preferably by a single injection, more preferably by intramuscular or sub-cutaneous injection; and optionally

(c) inseminating the non-human mammal or group of non-human mammals, preferably by artificial insemination, preferably near the time of ovulation and, more preferably, 1 to 4 days, even more preferably 36 to 48 hours after administration step (b).

This method is effective especially for groups of non-human mammals.

The invention may be used in non-human mammals and preferably in any ungulate, such as bovine, ovine, equine, sheep, or goats. The compositions and method of the invention are particularly effective for increasing fertility and/or fecundity and, especially, ovulation or pregnancy results or corpora lutea (CL) size in bovine.

### LEGEND TO THE FIGURES

FIG. 1: Synchronization Protocol used to evaluate different doses of rbLH on follicle dynamics (Study 1).

FIG. 2: Synchronization Protocol used to evaluate lower doses of rbLH on follicle dynamics (Study 2)

FIG. 3: Proportion of cows having premature ovulations following the use of differing gonadotropins. Premature ovulation was assumed for cows ovulating within 48 h after progesterone device removal. (Study 1)

FIG. 4: Dominant follicle growth rate (mm) measured by ultrasound from device removal to 24 h (Control, eCG 300 IU and 150 or 300 micrograms of rbLH. (Study 1)

FIG. 5: Area in mm² of the formed corpus luteum (CL) at 7 days after ovulation in cows treated with differing gonadotropins (Control, eCG 300 IU and 150 or 300 micrograms of rbLH). (Study 1)

FIG. 6: Proportion of cows having premature ovulations following the use of differing gonadotropins. Premature ovulation was assumed for cows ovulating within 48 h after progesterone device removal. (Study 2)

FIG. 7: Dominant follicle growth rate (mm) measured by ultrasound from device removal to 24 h (Control, eCG 300 IU and 50 or 100 micrograms of rbLH). (Study 2)

FIG. 8: Area in mm² of the formed corpus luteum (CL) at 7 days after ovulation in cows treated with differing gonadotropins (Control, eCG 300 IU and 50 or 100 micrograms of rbLH). (Study 2)

FIG. 9: Dominant follicle growth rate (mm) measured by ultrasound from device removal to 24 h (Control, eCG 300 IU and 50, 100, 150 or 300 micrograms of rbLH). (Studies 1 and 2)

FIG. 10: Ovulation rate at 72 h in cows treated with differing gonadotropins (Control, eCG 300 IU and 50, 100, 150 or 300 micrograms of rbLH). (Studies 1 and 2)

FIG. 11: Area in mm² of the formed corpus luteum (CL) at 7 days after ovulation in cows treated with differing gonadotropins (Control, eCG 300 IU and 50, 100, 150 or 300 micrograms of rbLH). (Studies 1 and 2)

### DETAILED DESCRIPTION OF THE INVENTION

The present invention provides methods to increase reproductive performance in non-human mammals, and preferably ungulates, even more preferably cattle. In particular, rLH is used at an efficient dose to stimulate fertility and/or fecundity in non-human mammal females, as illustrated by an increase of follicle growth rates. rLH is also effective to stimulate the growth and the maturation of follicles, to improve the ovulation rate, and to increase the size of corpora lutea (CL).

rLH can be used in synchronization protocols and/or treatments for fixed-time artificial insemination and/or embryo transfer. The invention may be used with any ungulate, preferably bovine.

### Definitions

Within the context of the present invention, the term “increasing reproductive performance” or “increased reproductive performance” refers to increasing the likelihood that a non-human mammal, or a plurality of non-human mammals, will be fertile and conceive.

Increasing reproductive performance includes a stimulation of the growth and/or maturation of follicles, or an improvement in the size (such as its surface area) of corpora lutea.

Increased reproductive performance also includes an increased likelihood that a non-human mammal, or a plurality of non-human mammals, which has been inseminated will become pregnant, will deliver a live offspring, or develop viable embryos.

Increasing reproductive performance also includes increasing the number of viable embryos. A non-human mammal or a plurality of non-human mammals can produce embryos in utero and/or in vitro. Increased reproductive performance includes increasing fertility, fecundity, superovulation, oocyte rate, ovulation rate, embryo production and/or pregnancies. An increase is preferably by approximately at least 1% as compared to non-treated non-human mammals, more preferably by at least 2%, 3%, 4%, 5%, 10% or more.

The term “fertility” or “fertile” refers, within the context of this invention, to the ability to produce fertilizable oocytes.

The term “fecund”, “conceive” or “fecundity” refers, within the context of this invention, to the ability to complete a pregnancy.

The term “superovulation” refers, within the context of this invention, to an increase in the number of ovulated follicles and/or in the creation of fertile ova.

The term “pregnant” refers to a non-human mammal or to a group of non-human mammals some of which being currently pregnant or that has been inseminated and may be pregnant.

As used herein, the term “estrus” refers to the period during which a non-human mammal is most likely to become pregnant. Estrus may be detected or monitored by behavioral demonstration that a non-human mammal is in heat, including showing standing heat.

“Insemination” refers to introducing semen by any method known in the art, including, but not limited to, natural and Artificial Insemination (AI) and in vitro fertilization (IVF).

A “group” of animals designates any group of at least 2 non-human mammals, such as a herd or flock.

An “ungulate” refers to any animal with hooves and especially the two taxonomic orders Perissodactyla and Cetartiodactyla.

The term “administration” refers to all route of administration such as oral, enteral mucosal, parenteral or percutaneous. Preferably the administration route is an injection, in particular intramuscular (IM) or subcutaneous (SC) injection.

As used herein, the term “about” or “around” will be understood by a person of ordinary skill in the art and will vary to some extent on the context in which it is used. If there are uses of the term which are not clear to persons of ordinary skill in the art given the context in which it is used, “about” or “around” will mean up to plus or minus 20%, preferably 10% or 5%, of the particular term.

### Recombinant Luteinizing Hormone and Use Thereof

The rLH may be produced in a baculovirus or mammalian or other expression system. In one embodiment, recombinant LH is recovered from the milk or egg whites of a transgenic animal. Methods of producing recombinant proteins in transgenic animals are well known and have been described in U.S. Pat. Nos. 4,873,316; 5,322,775; 6,111, 165; 6,472,584 and 6,528, 699 and other means known in the art.

Recombinant LH can be made using cloned and mutated LH genes that encode peptides identical to native LH, or having at least about 80% homology thereto, more preferably having at least about 90% homology thereto, and most preferably having at least about 95% homology thereto and also being able to induce ovulation in a mammal. Recombinant LH can also be made using cloned and mutated LH genes that encode peptides that are not identical to native LH, of the selected species, providing that the recombinant LH produced has a similar activity as native LH.

Recombinant LH can also be made in accordance with the methods known to the art, e. g., as described in US Patent Application No. 20030059898 assigned to Genzyme, and U.S. Pat. Nos. 6,635,256; 6,242,580; 6,238,890; 6,225,449; 6,103,501; 6,028,177; 5,985,611; 5,958,737; 5,883,073; 5,792,460; 5,759,818; 5,733,735; 5,712,122; 5,705,478; 5,585,345; 5,405,945; 5,338,835 and 5,177,193, and U.S. Patent Applications No. 20020160944, and 20010007757, and other means known to the art.

Production of recombinant bovine LH (rbLH) is described in WO 90/02757, U.S. Pat. Nos. 6,455,282; 5,639,639, 5,767,251, Nilson (1987) J. Reprod. Fertil. Suppl. 34: 227-36, Boime et al. (1992) Seminars in Reprod. Endocrin. 10: 45-50, and Kaetzel (1985) PNAS USA 82: 7280-7283. A process for the purification of recombinant LH is described in WO 01/62774. U.S. Pat. No. 5,929,028 describes liquid gonadotropin containing formulations that may include LH. Otieno et al. (2002 Reproduction 123 (1): 155-162) describes expression of LH genes in bovine conceptuses.

According to a preferred embodiment, the recombinant bovine luteinizing hormone analog (rbLH), comprises, or preferably consists of, a single polypeptide chain in which the alpha and beta subunits from bovine LH are covalently linked via a peptide linker. The peptide linker may be any peptide linker which does not affect the conformation or activity of LH. In a preferred embodiment, the linker is a CTP linker, e.g., a linker which comprises a sequence of the carboxy terminal peptide of human chorionic gonadotropin (hCG), as described in U.S. Pat. No. 6,242,580 and US2008/0312151. The rbLH used in the present invention are preferably as described in WO2004/078061. Single-chain recombinant bovine LH can be made in accordance with the methods described in U.S. Pat. No. 6,242,580, which discloses recombinant LH wherein the beta subunit is covalently linked to the alpha subunit. Alternatively, a linker is present between the beta and alpha subunits. Single-chain forms need only a single gene to be transcribed during recombinant production and are advantageous over the dimeric forms in terms of stability of the protein. Expression vectors where the C-terminus of the bovine beta subunit is preferably linked to the N-terminus of the bovine alpha subunit are transfected into CHO cells for expression.

According to a particular embodiment, rbLH comprises or consists of the SEQ ID NO: 2 or 3. SEQ ID NO: 1 presents a nucleotide expression sequence for rbLH SEQ ID NO: 3.

Recombinant LH is preferably used in essentially pure form, optionally in association with one or several pharmaceutically acceptable excipients or carriers. In a preferred embodiment, rLH is administered in a composition comprising a suitable pharmaceutical formulation. The pharmaceutical formulation may comprise one or several excipients or carriers.

The rLH is administered according to the invention at a dose range comprised between about 50 and about 300 micrograms per animal. More preferably the administered dose per animal is 50, 75, 100, 125, 150, 175, 200, 225, 250, 275, and 300 micrograms.

The rLH is preferably administered in one dose, i.e. in one single administration.

According to a particular embodiment, rLH is administered by injection, preferably by intramuscular or subcutaneous injection, and more preferably by intramuscular injection.

### Treatment

As previously indicated, the invention relates to novel methods for increasing reproductive performance in non-human mammals using rLH at a low dose. The invention may be used in insemination and/or embryo transfer synchronization programs particularly ungulates, e.g., to improve ovulation rate and/or follicle growth and/or the size of corpora lutea in the treated animals; as well as synchronization programs for embryo recipients, e.g., to improve pregnancy rate in recipients animals.

The invention is particularly suitable for timed-artificial insemination and/or embryo transfer protocols and/or treatments of non-human mammals, especially female beef and dairy cattle, including heifers.

More particularly, an object of the invention relates to rLH for use to increase reproductive performance in non-human mammals, wherein rLH is administered to said non-human mammals at a dose range comprised between about 50 and about 300 micrograms, preferably with a simultaneous administration of estradiol and/or PGF2a compound.

Another object of the invention resides in a method for increasing reproductive performance or embryo production in non-human mammals, comprising administering to said non-human mammals, rLH at a dose range comprised between about 50 and about 300 micrograms, preferably with a simultaneous administration of estradiol and/or PGF2a compound.

Another object of the invention resides in a method for increasing pregnancy rate in non-human mammals, comprising administering to said non-human mammal rLH at a dose range comprised between about 50 and about 300 micrograms, preferably with a simultaneous administration of estradiol and/or PGF2a compound.

Another object of the invention resides in a method for increasing follicle growth and ovulation rate in non-human mammals, comprising administering to non-human mammals, rLH at a dose range comprised between about 50 and about 300 micrograms, preferably with a simultaneous administration of estradiol and/or PGF2a compound.

In a particular object, the invention resides in a method for increasing follicle growth and ovulation rate in non-human mammals, comprising administering to non-human mammals, rLH at a dose range comprised between about 50 and about 100 micrograms, preferably with a simultaneous administration of estradiol and/or PGF2a compound

A further object of the invention is a method for improving corpus luteum quality in non-human mammals, comprising administering to a non-human mammal rLH at a dose range comprised between 50 and 300 micrograms, preferably with a simultaneous administration of estradiol and/or PGF2a compound.

In a particular object, the invention resides in a method for improving corpus luteum quality in non-human mammals, comprising administering to a non-human mammal rLH at a dose range comprised between 100 and 150 micrograms, preferably with a simultaneous administration of estradiol and/or PGF2a compound.

In a preferred embodiment, the method comprises:

(a) treating a non-human mammal (such as an ungulate) or a group of non-human mammals (such as a group of ungulates) for a timed-artificial insemination protocol and/or treatment, or providing a non-human mammal (such as an ungulate) or a group of non-human mammals (such as a group of ungulates) which has been treated for a timed-artificial insemination protocol and/or treatment;

(b) administering to the treated non-human mammal(s) rLH at a dose range comprised between about 50 and about 300 micrograms, in one single dose, and preferably with a simultaneous administration of estradiol and/or PGF2a compound; and

(c) optionally, inseminating the non-human mammal(s), preferably 1 to 4 days, or even more preferably 36 to 48 hours after administration step (b).

As mentioned before, utilization of synchronization programs, including the called “timed-artificial insemination” (TAI) protocols, comprise steps to synchronize ovulation in females, growth and ovulation of follicles in a synchronized fashion allowing the fixed-time artificial insemination, avoiding the necessity of estrus detection. As a first treatment step of synchronization programs, emergence of a new follicular wave is synchronized.

Follicular growth is not continuous in non-human mammals such as bovine, but occurs in waves (2 to 4 waves per cycle). Each wave begins approximately when the dominant follicle in the previous wave gains maximal size, at which time numerous small follicles begin a period of rapid growth. From this group of follicles, one follicle is allowed to grow to a much larger size than the others. This large follicle is called the dominant follicle, because it has the ability to regulate and restrict the growth of the smaller follicles, called subordinate follicles. A few days after reaching maximum size, the dominant follicle begins to regress and die. As the dominant follicle degenerates, its ability to restrict the other follicles is reduced; therefore, a new follicular wave is initiated. A consequence of this dynamic process is that follicles of all sizes, including at least one large follicle, exist on each day of the estrous cycle.

Follicle wave synchronization gives the opportunity to treat all non-human mammals in a limited period of time and, therefore, to capture the economic benefits of the insemination. Upon synchronization of the estrous cycle, a high percentage of treated females show a fertile, closely synchronized estrus and ovulation.

The synchronization of ovulation or timed-AI protocols refers to methods and/or protocols that artificially stimulate follicle growth and timed ovulation, so that ovulation is initiated at a predetermined time with no need to monitor estrus behavior. A review of common methods and protocol applied in bovine are described in the article of Bo et al in the 28^{th} Annual meeting AETE-Saint Malo, France, 7-8 Sep. 2012 (Recent advances in the control of follicular development and superovulation protocols in cattle).

A new follicle wave emergence can be synchronized by hormonal or physical treatment. Hormonal treatment comprises the administration of suitable hormones, such as estrogens and other hormones including progesterone or GnRH. Physical treatment includes follicle ablation.

In a preferred embodiment, prior to rLH administration according to the invention, ovulation synchronization (or step a) is carried out and comprises a hormonal treatment of the non-human mammal(s), preferably by administration of GnRH and/or estrogen-progesterone combination.

In a preferred embodiment, step a is carried out by implementing the following treatment: simultaneous administration of estrogen compound and progesterone. Preferably, estrogen is administered by injection, preferably by intramuscular injection. Preferably, progesterone is administered intravaginally via a device, such as an implant (also called P4 device in the following examples).

According to a particular embodiment, the progesterone device is maintained from 5 to 10 days, preferably from 7 to 9 days, and is removed thereafter, and more particularly from 8 to 9 days (e.g. the device is removed 8.5 days after insertion).

Specific dosages and/or protocols are disclosed in the art such as, e.g., in Thatcher et al., 2001 (American Association of Bovine Practitioner, AABP, Vancouver, 95-105); Diskin et al., 2001 (occasional publication n° 26, p175, British society of Animal Science); or Pursley et al., 1995 (Theriogenology 44 p915).

According to a particular embodiment, rLH is administered according to the invention at the device removal.

In a preferred embodiment, the invention relates to a method comprising:

(a) treating a non-human mammal (such as an ungulate) or a group of non-human mammals (such as ungulates) with a GnRH and/or progesterone-estrogen combination to synchronize follicles; or providing a non-human mammal (such as an ungulate) or a group of non-human mammals (such as ungulates) which has been treated with a GnRH and/or progesterone-estrogen combination to synchronize follicles;

(b) administering to the treated non-human mammals (such as ungulates) rLH at a dose range comprised between about 50 and about 300 micrograms, preferably with a simultaneous administration of estradiol and/or PGF2a compound; and

(c) optionally, inseminating a treated non-human mammal (such as ungulate) of (b) and/or collecting oocytes from a treated non-human mammal (such as ungulate) of (b), preferably near the time of ovulation and, more preferably, within 1 to 4 days, even more preferably 36 to 48 hours after administration step (b).

In step (b), rLH can be administered using any means or techniques known per se in the art including, without limitation, systemic administration, such as intramuscular, intravenous, subcutaneous, etc. Preferred administration route is intramuscular injection.

In a preferred embodiment, rLH is administered in one single dose.

In a preferred embodiment, estrogen is an estradiol compound. The estradiol compound is preferably an estradiol ester, including estradiol cypionate, valerate or benzoate. According to a more preferred embodiment, the estradiol ester is estradiol benzoate. According to a particular embodiment, rLH is administered at a dose range comprised between about 50 and about 300 micrograms, with a simultaneous administration of estradiol benzoate (EB) or estradiol cypionate.

According to a particular embodiment, rLH is administered simultaneously with prostaglandin, more specifically prostaglandin F2alpha (PGF2a). According to a more particular embodiment, rLH is administered simultaneously with estradiol compound (preferably estradiol benzoate) and prostaglandin (preferably PGF2a).

In a preferred embodiment, the method further comprises a step (c) of inseminating said ungulate, preferably near the time of ovulation and, more preferably, within 1 to 4 days, even more preferably 36 to 48 hours after rLH administration.

The prostaglandin, as well as estradiol compound, are administered typically by injection, as a single or multiple dose(s), more preferably each as a single dose.

The invention may be used in any ungulate, such as bovine, sheep, goats, cervids, yaks, water buffaloes, bison, antelopes, gazelles, elk, reindeer, moose, bighorn sheep, giraffes, camelids, swine, equine, alpacas, and vicunas. It is particularly suited for treating female beef and dairy cattle, including heifers.

SEQUENCE LISTING

```
SEQ ID NO 1
atggagatgttccagggactgctgctgtggctgctgctgggcgtggccggggtg
  M E M F Q G L L L W L L L G V A G V
tgggcttccagggggccactgcggccgctgtgccagcccatcaacgccaccctg
  W A S R G P L R P L C Q P I N A T L
gcggctgagaaggaggcctgccctgtctgtatcactttcaccaccagcatctgc
  A A E K E A C P V C I T F T T S I C
gccggctactgccccagcatgaagcgggtgctgcctgtcatcctgccgcccatg
  A G Y C P S M K R V L P V I L P P M
ccccagcgggtgtgcacctaccatgagctgcgcttcgcctccgttcggctcccc
  P Q R V C T Y H E L R F A S V R L P
ggctgcccacctggagtggacccaatggtctccttccccgtggccctcagctgt
  G C P P G V D P M V S F P V A L S C
cactgtggaccctgccgcctcagcagcactgactgcgggggtcccagaacccaa
  H C G P C R L S S T D C G G P R T Q
cccttggcctgtgaccaccccccgtcctcttcctcaaaggcccctcccccgagc
  P L A C D H P P S S S S K A P P P S
cttccaagtccatcccgactcccggggccctcggacaccccgatcctcccacaa
  L P S P S R L P G P S D T P I L P Q
tttcctgatggagagtttacaatgcagggctgtcctgaatgcaagctaaaagaa
  F P D G E F T M Q G C P E C K L K E
aacaaatacttctccaagccagatgctccaatctatcagtgcatggggtgctgc
  N K Y F S K P D A P I Y Q C M G C C
ttctccagggcataccccactccagcgaggtctaagaagacaatgttggtcccc
  F S R A Y P T P A R S K K T M L V P
aagaacatcacctcggaagctacatgctgtgtggccaaagcatttaccaaggcc
  K N I T S E A T C C V A K A F T K A
acagtgatgggaaatgtcagagtggagaaccacaccgagtgccactgcagcact
  T V M G N V R V E N H T E C H C S T
tgttattatcacaaatcctaa
  C Y Y H K S *
1st bold sequence: signal peptide
1st non-bolded sequence: Beta subunit
2nd bold sequence: CTP linker
2nd non-bolded sequence: Alpha subunit
SEQ ID NO 2
S R G P L R P L C Q P I N A T L A A E K E A C P V
C I T F T T S I C A G Y C P S M K R V L P V I L P
P M P Q R V C T Y H E L R F A S V R L P G C P P G
V D P M V S F P V A L S C H C G P C R L S S T D C
G G P R T Q P L A C D H P P S S S S K A P P P S L
P S P S R L P G P S D T P I L P Q F P D G E F T M
Q G C P E C K L K E N K Y F S K P D A P I Y Q C M
G C C F S R A Y P T P A R S K K T M L V P K N I T
S E A T C C V A K A F T K A T V M G N V R V E N H
T E C H C S T C Y Y H K S
SEQ ID NO 3
M E M F Q G L L L W L L L G V A G
V W A S R G P L R P L C Q P I N A T L A A E K E
A C P V C I T F T T S I C A G Y C P S M K R V L P
V I L P P M P Q R V C T Y H E L R F A S V R L P G
C P P G V D P M V S F P V A L S C H C G P C R L S
S T D C G G P R T Q P L A C D H P P S S S S K A P
P P S L P S P S R L P G P S D T P I L P Q F P D G
E F T M Q G C P E C K L K E N K Y F S K P D A P I
Y Q C M G C C F S R A Y P T P A R S K K T M L V P
K N I T S E A T C C V A K A F T K A T V M G N V R
V E N H T E C H C S T C Y Y H K S
```

Further aspects and advantages of the invention will be disclosed in the following experimental section, which illustrates the claimed invention.

### EXAMPLES

### Methods

A series of several studies were performed by implementing rbLH (in particular rbLH comprising SEQ ID NO: 2) during timed AI protocols in Bos indicus (Nelore) cattle, and comparison with a control or eCG treatment.

Study 1—Evaluation of Different Doses of rbLH on Follicle Dynamics.

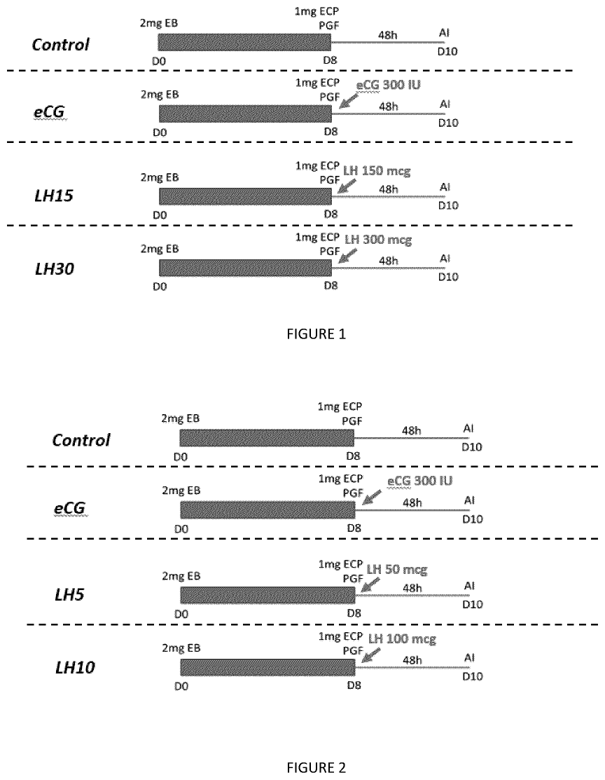

Thirty-nine healthy postpartum anestrus Nelore cows, weighing between 400 to 600 kg and ages of 2 to 8 years old were included in the trial. Cows were kept in Brachiaria pastures and were identified by ear tag number. All animals received a progesterone device (P4 device—loaded with 750 mg of natural progesterone) on DO plus 2 mg of estradiol benzoate (EB). Eight days later, at device removal, all animals received a PGF2a (PGF) treatment simultaneously to 1 mg of estradiol cipionate (ECP), and at the same time were randomized (CRD—complete randomized design) to receive one of the 4 treatments, as follows: eCG (300 IU of eCG, n=11); Negative Control (no gonadotropin treatment, n=9); rbLH15 (150 micrograms of rbLH, n=9) and rbLH30 (300 micrograms of rbLH, n=10). This group of cows undergoing the follicular dynamics evaluations received frequent ultrasound scannings (US) throughout the synchronization protocol to evaluate primarily the proportion of cows ovulating within 48 h after device removal as well as follicle growth following the differing treatments. FIG. 1

Study 2—Evaluation of a Lower Dose of rbLH on Follicle Dynamics.

Sixty-two healthy postpartum anestrus Nelore cows, weighing between 400 to 600 kg and ages of 2 to 8 years old were included in the trial. Cows were kept in Brachiaria pastures and were identified by ear tag number. All animals received a progesterone device on DO plus 2 mg of estradiol benzoate (EB). Eight days later, at device removal, all animals received a PGF2a treatment simultaneously to 1 mg of estradiol cypionate (ECP), and at the same time were randomized (CRD—complete randomized design) to receive one of the 4 treatments, as follows: eCG (300 IU of eCG, n=12); Negative Control (no gonadotropin treatment, n=14); rbLH5 (50 micrograms of rbLH, n=16) and rbLH10 (100 micrograms of rbLH, n=20). This group of cows undergoing the follicular dynamics evaluations received frequent ultrasound scanning throughout the synchronization protocol to evaluate primarily the proportion of cows ovulating within 48 h after device removal as well as follicle growth following the differing treatments. FIG. 2

### Measurements

Initial body condition scores, days post-partum and parity number of the cows;
Ultrasound examinations on DO, D 8 to D10 at 12 h intervals to evaluate follicular dynamics during the synchronization protocol;
Follicle growth, follicle size at 48 h and time of ovulation after device removal.

### Statistics

Data were analyzed by logistic regression using the GLIMMIX procedure of SAS (version 9.4). The final logistic regression model removed variables by a backward elimination based on the Wald statistics criterion when p>0.20. Statistical significance was assumed when P-value was lower than 0.05 and statistical differences are indicated with differing superscript letters. All values are presented as LSmeans extracted from the ILINK option from the GLIMMIX procedure of the SAS software.

### Results

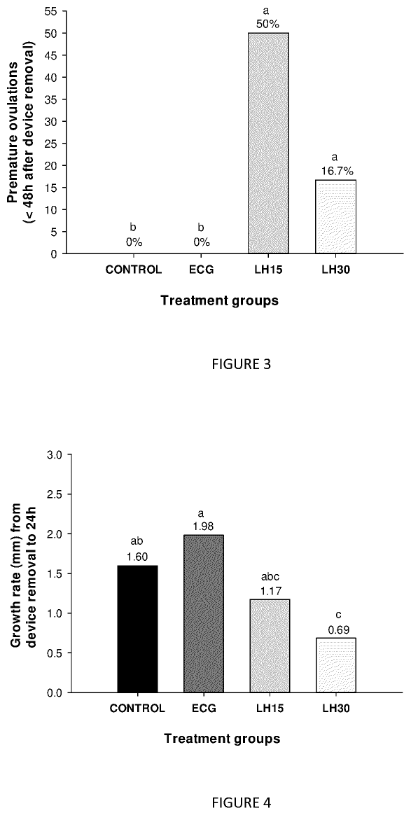

When testing different doses of rbLH as alternative to eCG in a synchronization protocol for TAI, the results from Study 1 showed a large proportion of cows ovulating prematurely following the use of rbLH compared to eCG and Control groups (FIG. 3). Also the growth rate of the dominant follicle from device removal to 24 h was much lower for cows receiving rbLH compared to Control cows and cows receiving 300 IU of eCG (FIG. 4). However, the results presented in FIG. 5 show an equivalent CL area for cows treated with 300 IU of eCG and 150 or 300 micrograms of rbLH, and both groups produced CL structures that were significantly greater than untreated Control cows.

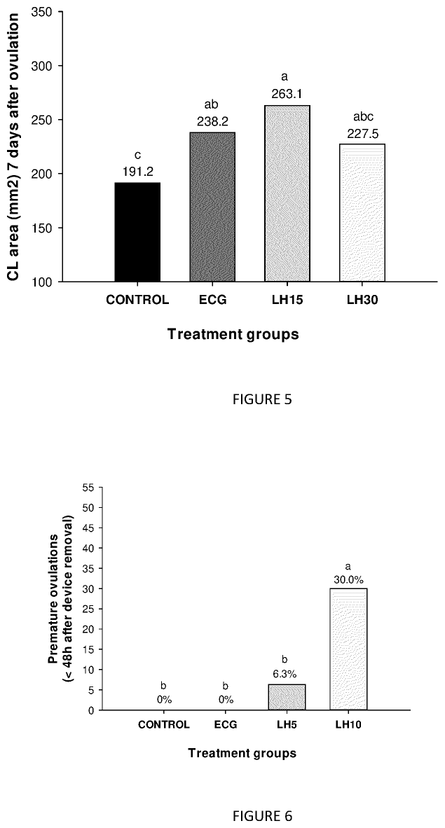

Based on the satisfactory results on CL areas for cows treated with 150 or 300 micrograms of rbLH, it was decided to repeat Study 1 using a lower dose of rbLH (50 and 100 micrograms) in the attempt to replace eCG. As shown in FIG. 6, the dose of 50 micrograms of rbLH did not induce premature ovulations, and only 7% of cows receiving 50 micrograms of rbLH ovulated before 48 h after device removal, whereas, at the dose of 100 micrograms of rbLH, 30% of cows had premature ovulation.

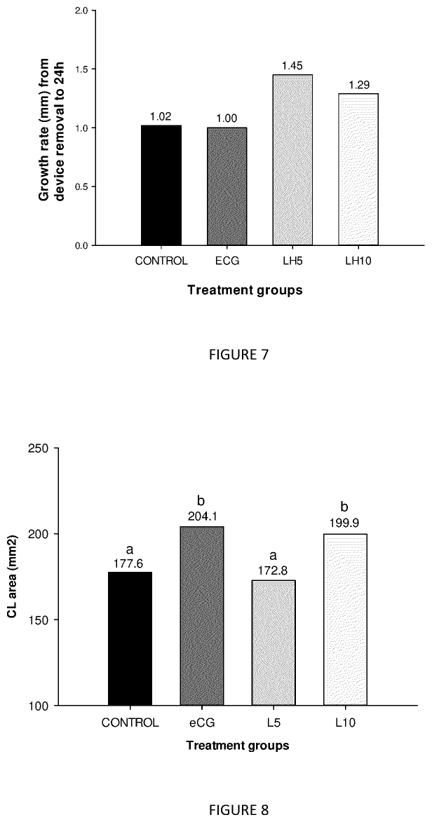

Dominant follicle growth rate from device removal to 24 h was higher for cows receiving 50 and 100 micrograms of rbLH compared to Control cows and cows receiving 300 IU of eCG (FIG. 7), whereas CL area for cows treated with 300 IU of eCG and 100 micrograms of rbLH was significantly greater than untreated Control cows and for cows receiving 50 micrograms of rbLH.

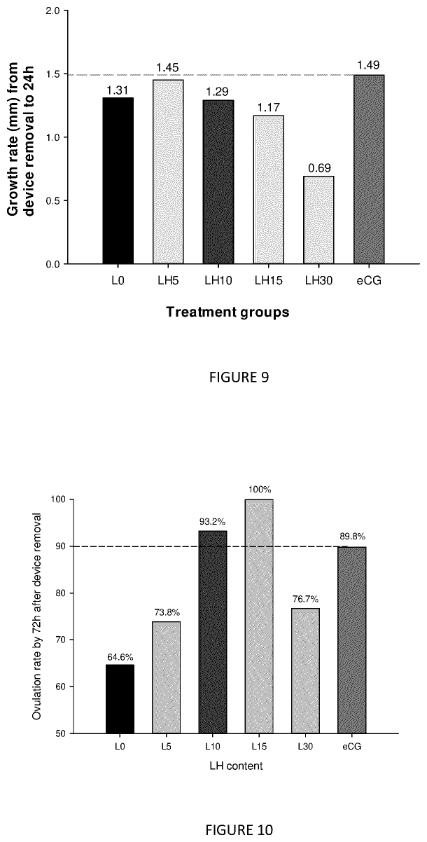

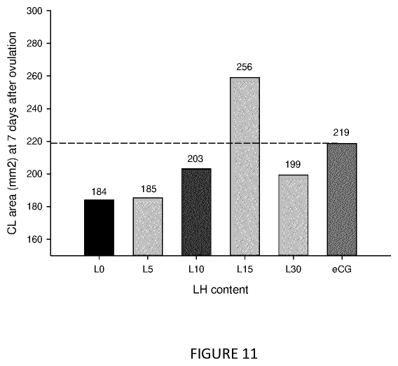

When combining the two experiments, although the greater incidence of premature ovulation rate compared to eCG, cows treated with rbLh at the dose of 100 and 150 micrograms achieved similar follicular growth (FIG. 9), similar ovulation rate at 72 h (FIG. 10) and CL area (FIG. 11) as cows treated with eCG and significantly higher than negative Control group cows.

An additional study is planned to test follicular growth and ovulation time using the dose of 100 and/or 150 micrograms of rbLH with estradiol benzoate used to induce ovulations that match the circulating half-life (˜30 h) of rbLH. After testing the best rbLH dose on follicular growth, a large field study will be conducted to test fertility outcomes of rbLH as a replacement to eCG in a synchronization protocol for timed artificial insemination (TAI) in cattle.

### CONCLUSIONS

The use of rbLH, in particular 100 or 150 micrograms rbLH, at final stages of the synchronization protocol for timed-AI:
Improved follicle growth rates comparable to eCG;
Increased CL area 7 days after ovulation, which is critical for embryo development.

1. A method of increasing reproductive performance in a non-human mammal, the method comprising administering recombinant luteinizing hormone or a composition comprising recombinant luteinizing hormone to a non-human mammal at a dose range of between about 50 and about 300 micrograms.

2. The method of claim 1, wherein recombinant luteinizing hormone is administered in one single administration.

3. The method of claim 1, wherein said recombinant luteinizing hormone is administered by an injection.

4. The method of claim 1, wherein recombinant luteinizing hormone is administered simultaneously with prostaglandin.

5. The method of claim 1, wherein recombinant luteinizing hormone is administered simultaneously with estradiol compound and prostaglandin.

6. The method of claim 1, wherein, prior to recombinant luteinizing hormone administration, estrogen and progesterone are administered to the non-human mammal.

7. The method of claim 6, wherein estrogen is administered by injection.

8. The method of claim 5, wherein progesterone is administered intravaginally via a device.

9. The method of claim 1, further comprising inseminating said non-human mammal near the time of ovulation.

10. The method of claim 1, comprising:
(a) providing a non-human mammal or a group of non-human mammals, which has been treated to synchronize follicles;
(b) administering to said non-human mammal or group of non-human mammals recombinant luteinizing hormone at a dose range comprised between about 50 and about 300 micrograms; and
(c) optionally inseminating the non-human mammal or group of non-human mammals, or collecting oocytes from the non-human mammal or group of non-human mammals.

11. The method of claim 1, comprising:
(a) treating a non-human mammal, or a group of non-human mammals, to synchronize follicles;
(b) administering to said non-human mammal or group of non-human mammals recombinant luteinizing hormone at a dose range comprised between about 50 and about 300 micrograms; and
(c) optionally inseminating the non-human mammal or group of non-human mammals, or collecting oocytes from the non-human mammal or group of non-human mammals.

12. The method of claim 10, wherein follicle synchronization is obtained by hormonal treatment of the non-human mammal.

13. The method of claim 1, wherein the non-human mammal is an ungulate.

14. The method of claim 1, wherein the reproductive performance is increased by increasing pregnancy in the non-human mammal.

15. The method of claim 1, wherein the reproductive performance is increased by increasing ovulation rate in said non-human mammal.

16. The method of claim 1, wherein the recombinant bovine luteinizing hormone analog (rbLH), comprises a single polypeptide chain in which the alpha and beta subunits from bovine LH are covalently linked via a peptide linker.

17. The method of claim 1, wherein the method further comprises a simultaneous co-administration of estradiol and/or PGF2alpha compound.

18. The method of claim 2, wherein the method further comprises a simultaneous co-administration of estradiol benzoate and/or PGF2alpha compound.

19. The method of claim 3, wherein the injection is an intramuscular or a subcutaneous injection.

20. The method of claim 19, wherein the injection is an intramuscular injection.


SEQUENCE LISTING (3 sequences)

SEQ ID NO: 1 - DNA, 777 bp, Artificial
  recombinant bovine luteinizing hormone
atggagatgttccagggactgctgctgtggctgctgctgggcgtggccggggtgtgggcttccagggggccactgcggccgctgtgccagcccatcaacgccaccctggcggctgagaaggaggcctgccctgtctgtatcactttcaccaccagcatctgcgccggctactgccccagcatgaagcgggtgctgcctgtcatcctgccgcccatgccccagcgggtgtgcacctaccatgagctgcgcttcgcctccgttcggctccccggctgcccacctggagtggacccaatggtctccttccccgtggccctcagctgtcactgtggaccctgccgcctcagcagcactgactgcgggggtcccagaacccaacccttggcctgtgaccaccccccgtcctcttcctcaaaggcccctcccccgagccttccaagtccatcccgactcccggggccctcggacaccccgatcctcccacaatttcctgatggagagtttacaatgcagggctgtcctgaatgcaagctaaaagaaaacaaatacttctccaagccagatgctccaatctatcagtgcatggggtgctgcttctccagggcataccccactccagcgaggtctaagaagacaatgttggtccccaagaacatcacctcggaagctacatgctgtgtggccaaagcatttaccaaggccacagtgatgggaaatgtcagagtggagaaccacaccgagtgccactgcagcacttgttattatcacaaatcctaa

SEQ ID NO: 2 - protein, 238 aa, artificial
  recombinant bovine luteinizing hormone (without signal peptide)
SRGPLRPLCQPINATLAAEKEACPVCITFTTSICAGYCPSMKRVLPVILPPMPQRVCTYHELRFASVRLPGCPPGVDPMVSFPVALSCHCGPCRLSSTDCGGPRTQPLACDHPPSSSSKAPPPSLPSPSRLPGPSDTPILPQFPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 3 - protein, 258 aa, artificial
  recombinant bovine luteinizing hormone (with signal peptide)
MEMFQGLLLWLLLGVAGVWASRGPLRPLCQPINATLAAEKEACPVCITFTTSICAGYCPSMKRVLPVILPPMPQRVCTYHELRFASVRLPGCPPGVDPMVSFPVALSCHCGPCRLSSTDCGGPRTQPLACDHPPSSSSKAPPPSLPSPSRLPGPSDTPILPQFPDGEFTMQGCPECKLKENKYFSKPDAPIYQCMGCCFSRAYPTPARSKKTMLVPKNITSEATCCVAKAFTKATVMGNVRVENHTECHCSTCYYHKS